(19) United States
(12) Patent Application Publication
(10) Pub. No.: US 2012/0058048 A1
(43) Pub. Date: Mar. 8, 2012

(54) DIRECTED RADIOTHERAPY

(76) Inventor: William Herman, Thornhill (CA)

(21) Appl. No.: 12/876,930

(22) Filed: Sep. 7, 2010

(51) Int. Cl. A61K 51/00; A61K 39/395; A61P 35/00; A61K 9/14

(52) U.S. Cl. 424/1.29; 424/490; 424/136.1

(57) ABSTRACT

The present invention relates enhanced targeting of drug delivery vehicles to vascular endothelial cells

### BACKGROUND OF THE INVENTION

1. Field of the Invention

The present invention relates enhanced targeting of drug delivery vehicles to vascular endothelial cells

2. Description of the Prior Art

Individualized dosing for radiotherapy including therapies in which radionuclides are administered, for example by way of targeted radioligands and nanoparticles, has been advocated for enhancing safety and effectiveness of the therapeutic regimen. Monitoring can be done via imaging studies, for example, MRI and other non-invasive an invasive techniques. It has been advocated that dosimetry for radionuclide treatment be individualized (see Stabin M G. Update: the case for patient-specific dosimetry in radionuclide therapy. Cancer Biother Radiopharm. 2008 June; 23(3):273-84. Stabin M G. Uncertainties in internal dose calculations for radiopharmaceuticals. J Nucl Med. 2008 May; 49(5):853-60.)

Therapeutic encapsulation has been used to control the delivery of therapeutics to a desired site in need thereof by controlling the properties of the polymer encapsulating the therapeutic to satisfy particular needs, such as degradability, swelling, permeability, temperature and pH sensitivity. Encapsulation has been accomplished through various ways, such as the use of microspheres/microparticles and nanomaterials. For example, U.S. Pat. No. 4,861,627 to Mathiowitz and Langer. discloses a single step method for preparation of multilayer polymeric drug delivery devices. U.S. Pat. No. 5,500,161 to Andrianov and Langer discloses a method for the preparation of microparticles, and the product thereof, that includes dispersing a substantially water insoluble non-ionic or ionic polymer in an aqueous solution in which the substance to be delivered is also dissolved, dispersed or suspended, and then coagulating the polymer together with the substance by impact forces to form a microparticle. Other patents and applications by Langer disclosing microparticles include U.S. Pat. Nos. 5,543,158; 5,578,325; 5,654,381; 5,718,921; 5,912,097; 6,007,845; 6,254,890; 6,800,296; 6,855,329; 6,998,115; 2002/0131951; 2003/0118692; 2004/0071654; and 2005/0123596. Spray drying has also been used to create drug-encapsulated microparticles.

The delivery of radiotherapy to tumor vasculature is conventionally accomplished with antibodies conjugated to radionuclides, for example, by pre-targeting with bispecific antibodies and delivering radiolabeled haptens (Reilly R M; Goldenberg D M, et al.). A variety of targeted nanomaterials are also being used as vehicles for this purpose, including liposomes loaded with alpha-emitters (Escorcia F E, et al.; Sofou S, et al.).

Liposomes are colloidal structures of lipid bilayers surrounding a central aqueous space, wherein a drug can be included. (Wang X, et al.) Several examples of liposome drugs include stealth liposomal doxorubicin (DOXIL®, Ortho Biotech), liposomal doxorubicin (MYOCET®, Sopherion Therapeutics, Inc.), and liposomal daunorubicin (DAUNOXOME®, Diatos), which are approved for treatment of breast cancer and Kaposi's sarcoma. Liposomes have been made to be targeted to tumors by conjugating a targeting moiety on the outer surface of the lipid bilayer. Studies have shown that this increases uptake of the liposomes and thus also the therapeutic enclosed.

Polymer-based nanoparticles have also been used to overcome the problem of microparticles being caught in the mononuclear phagocytic system (MPS) and being rapidly cleared from the body. A hydrophilic coating can enhance biocompatibility by protecting from capture by macrophages and enzymatic degradation. Several polymeric anticancer formulations are also available, such as paclitaxel poliglumex.

Passive and active targeting can be used to target nanoparticles to tumors. Passive targeting utilizes the properties of the tumor vasculature and microenvironment. For example, tumors have a unique effect known as enhanced permeability and retention (EPR) due to gaps in angiogenic blood vessels. This effect allows nanoparticles to enter the tumor vasculature. Active targeting utilizes a targeting ligand or antibody conjugated to the nanoparticle which can specifically bind to receptors or antigens expressed on the tumor. For example, galactosamine moieties on a HPMA copolymer-DOX-galactosamine nanoparticle has been shown to bind to the asialoglycoprotein receptor on hepatocytes. (Wang X, et al.) Receptors and antigens can be chosen that are uniquely or overexpressed on tumor cells, but are not expressed or have negligible expression on normal cells.

There remains a need for an effective cancer treatment with nanoparticles or microspheres.

### SUMMARY OF THE INVENTION

In one aspect the invention is directed to a heterofunctional ligand for enhancing the uptake in solid tumors of anti-neoplastic agents (including without limitation, nanoparticles, targeted biopharmaceuticals including antibodies and fragments thereof carrying a payload and small molecules chemotherapeutics of any kind, for example molecules that cause DNA damage or inhibit transcription (for example anthracyclines) In one embodiment, the heterofunctional ligand enhances the cell surface expression of molecules which interact with immune cells by enabling them to roll on the neo-vasculature of solid tumors. In one embodiment, the heterofunctional ligand enhances perivascular perfusion in solid tumors—tumor penetration. In one aspect, the present invention is directed to a heterofunctional ligand comprising a ligand or moiety that binds specifically to a target that is predominantly expressed (i.e. advantageous for selective of preferential targeting) on the intra-luminal surface of tumor vasculature (hereafter optionally referred to as neo-vascular targets and neo-vasculature targeting ligands, respectively), for example VEGFR-1 or VEGFR-2, endoglin, alphaVbeta3 etc. (for example an antibody that specifically recognizes such a target—the term “antibody” and “bispecific antibody” includes functional fragment thereof and varied constructs well known to those skilled in the art) The neo-vascular targeting ligand is linked (optionally in a manner suited for receptor cross-ligation), for example, via a flexible linker (for example, linkers of 15 to 30 amino acids consisting primarily of glycine, for example linkers conventionally used to link the VH and VL of single chain antibodies) to a ligand expressed on endothelial cells that binds to endothelial cells and enhances pericellular perfusion and/or expression of a ligand that mediates rolling of immune cells on endothelial cells, for example, CD62e and/or CD62p, for example TNF-α and particularly a mutant form thereof with lower affinity for TNFR-1 and TNFR-2, or TNFR-1 alone, that retains its agonist properties, or an interleukin, for example IL-1β. In one embodiment, the ligand that enhances expression of CD62e and/or CD62p. In one embodiment the ligand exerts an agonist effect on TNFR-1 and/or TNFR-2, in particular TNFR-1, for example a mutant TNF-α or a TNFR-1 agonist antibody. In one embodiment, is expressed on the intra-luminal surface of tumor vasculature co-localizes with TNFR1 or 2 on the surface of ne-vascular endothelial cells, for example in calveoli. In one embodiment, neo-vascular targeting ligand is VEGF A or an anti-VEGFR antibody construct or fragment, including common splice variant of VEGF A such as VEGF 165. In one embodiment the n-terminus of a TNF-α mutant is linked to the c-terminus of a splice variant of VEGF A via a flexible amino acid linker.

In one aspect the invention is directed to use of a heterofunctional ligand comprising the n-terminus of a TNF-α mutant linked to the c-terminus of a splice variant of VEGF A or a mutant thereof and a flexible amino acid linker for enhancing the tumor uptake of anti-neoplastic agents. In one embodiment, the affinity of this mutant is adapted for treatment of a solid tumor in a mammal. In one embodiment, the reduction in affinity of this mutant is adapted for treatment of an individual mammal, based on the cell surface expression of neo-vascular targets and TNFR-1 molecules, and the affinity of the neo-vascular targeting ligand. In one embodiment the affinity of the neo-vascular targeting ligand for the neo-vascular target is in the low nanomolar range (0.1 to 10 nanomolar) and the affinity of TNF-α agonist mutant is 5 to 1000 fold less. In one embodiment, the TNF-α agonist mutant is used for isolated perfusion and is optionally adapted via isolated perfusion, for example isolated limb, tissue or organ perfusion and binds to TNFR-1 with an affinity that is 5-100 fold less than that of the VEGF A moiety for VEGFR-2. In one embodiment, the isolated tissue is treated with a combination of radiotherapy using a targeted ligand or nanoparticle and chemotherapy.

In another aspect the present invention is directed to a microsphere coated with rolling ligands and carrying a radionuclide or agent to facilitate imaging, for example a contrast agent or fluorescent molecule.

In one embodiment, the microsphere carries a therapeutic radionuclide and is optionally adapted to serve a contrast agent.

In one embodiment the microsphere is adapted for removal from blood after intravascular administration. In one embodiment, the microsphere (which may be referred to as a rolling microsphere for convenience) is paramagnetic or superparamagnetic.

The term microsphere is used for convenience to broadly refer to a nanoparticle that is adapted for diagnostic or therapeutic use or use as a tool in animal research in the sense that it is at least suitable for administration into the vasculature of a mammal for example into an limb, organ or tissue the circulation of which is isolated. In one aspect, the invention is directed to nanoparticles that are adapted for rolling by being coated with rolling ligands and hence the term microsphere is used for convenience without reference to a particular size other than its ability to pass through capillaries unimpeded by its intrinsic size (less than approximately 5 microns for adults humans).

In one aspect the invention is directed to a system, use or method of using a microsphere as tool for research in animals, or as diagnostic, theragnostic or therapeutic, for diagnosing or treating a disease in a mammal, for example a solid tumor, wherein the microsphere carries a radionuclide therapeutic and ligands that mediate rolling on endothelium that exhibits an inflammatory response in the nature of enhanced cell surface expression of ligands that mediate rolling of immune cells, for example selectins. In one embodiment the ligands that mediate rolling are those over-expressed on the neo-vasculature of certain tumors. For convenience, these ligands that mediate rolling may be referred herein as “rolling ligands” and their corresponding ligands on endothelial cells as “rolling ligand receptors”. In one embodiment, the use, system or method comprises an immune modulator for increasing the endothelial surface expression of rolling ligand receptors and/or increasing the vascular permeability of a diseased tissue, for example a solid tumor. Optionally, the immune modulator acts directly on the endothelium. Optionally, the immune modulator is a TNFR agonist. Optionally, the immune modulator is a heterofunctional ligand comprising a TNFR agonist.

Accordingly, the invention is also directed to a composition of matter comprising a heterofunctional ligand including a neo-vascular targeting ligand and an TNFR-1 or TNFR-2 agonist. Optionally the neo-vascular targeting ligand colocalizes with the TNFR agonist, optionally as demonstrated by FRET, co-immunoprecipitation or a competition binding experiment well known to those skilled in the art, for example where the neo-vascular targeting ligand component of the heterofunctional ligand enhances the ability of the TNFR agonist (relative to when the agonist is unlinked to the vascular targeting ligand) to compete for binding with a test ligand (i.e when the two ligands are linked in a manner suitable for cross-ligation of the targets an avidity effect is demonstrated by enhanced competition) Optionally, the heterofunctional ligand is a bispecific antibody. Optionally, the agonist is a TNFR-1 agonist having an affinity for TNFR-1 that is 10 to 5000 fold lower than wild-type TNFα.

In one aspect, the invention is directed to a composition of matter comprising the rolling microsphere described above and a pharmaceutically acceptable carrier suited for parenteral administration.

In one aspect, the invention is directed to a composition of matter comprising heterofunctional ligand described above and a pharmaceutically acceptable carrier suited for parenteral administration.

In one aspect, the invention is directed to microsphere coated with ligands that bind to CD62e and/or CD62p and a method of enhancing the delivery of nanoparticles or microspheres that are coated with ligands that bind to CD62e and/or CD62p. Optionally, the nanoparticles or micropsheres are also coated with ligands that bind to targets that are predominantly expressed on the intra-luminal surface of tumor vasculature. The nanoparticles or microspheres may further include a therapeutic coated thereon, for example a radionuclide, and/or encapsulated therein.

The present invention further provides for a pharmaceutical or diagnostic composition of the aforesaid microspheres in a pharmaceutically acceptable carrier, wherein the surface of the microsphere comprises a coupling moiety for coupling to a radionuclide. In one embodiment the microphere is functionalized for loading rolling ligands and a therapeutic radionuclide. In one embodiment, the microsphere further comprises an imaging agent, namely a component that facilitates tracking the course a pharmaceutical composition comprising the rolling microsphere through a diseased tissue, for example a solid tumor.

In one aspect, the invention is directed to a method of using microspheres which are adapted to encapsulate or have coupled to its surface to at least one imaging agent and optionally a radionuclide, comprising the steps of:

loading onto the surface of the microspheres a quantity of rolling ligands which enable the microspheres to roll in a diseased tissue;

parenterally administering the microspheres to a mammal having or suspected of having a site of “enhanced” endothelial expression of rolling ligands receptors;

monitoring the distribution of the microspheres to the site of enhanced expression of rolling ligand receptor using a imaging apparatus, optionally, an apparatus adapted for MRI, and optionally one or more other regions of the body, optionally especially in the case of systemic administration, sites of possible rolling normally anticipated to engender toxic side effects e.g. major organs, bone marrow, spleen etc.

In one embodiment of the method consists essentially of step 2 and 3 and optionally other steps mentioned hereafter which are carried out with microspheres pre-loaded with rolling ligands.

The term “enhanced” with reference to endothelial expression of rolling ligand receptors, means endothelium in blood vessel sites which are not in an inflammatory state correlated with such enhanced expression.

Monitoring is optionally additionally carried out in blood which are not expected to be affected by the administration or are of particular concern e.g if systemically the vessels of major organs and/or bone marrow or e.g. with respect to isolated perfusion, the non-targeted tissue in the isolated tissue).

In one embodiment, the quantity of rolling ligands is approximately matched to an expected or estimated quantity of rolling ligand receptors on the surface of endothelial cells. The method optionally comprises the step of using an experimental apparatus and/or a mathematical model and/or animal model to predict the approximate rate of rolling of the sphere, for example a parallel plate flow chamber or other similar device and algorithms well known to those skilled in the art.

### DETAILED DESCRIPTION OF THE DRAWINGS

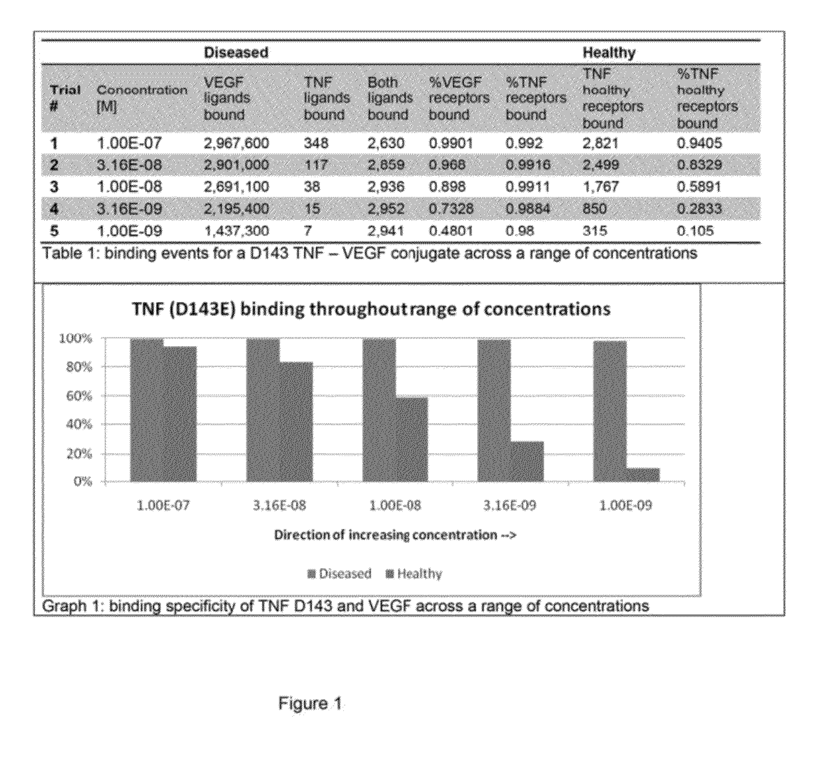

FIG. 1 is a table (Table 1) and accompanying graph (Graph 1) presenting calculated binding events for a D143 TNF-VEGF conjugate across a range of concentrations of D143 TNF-VEGF

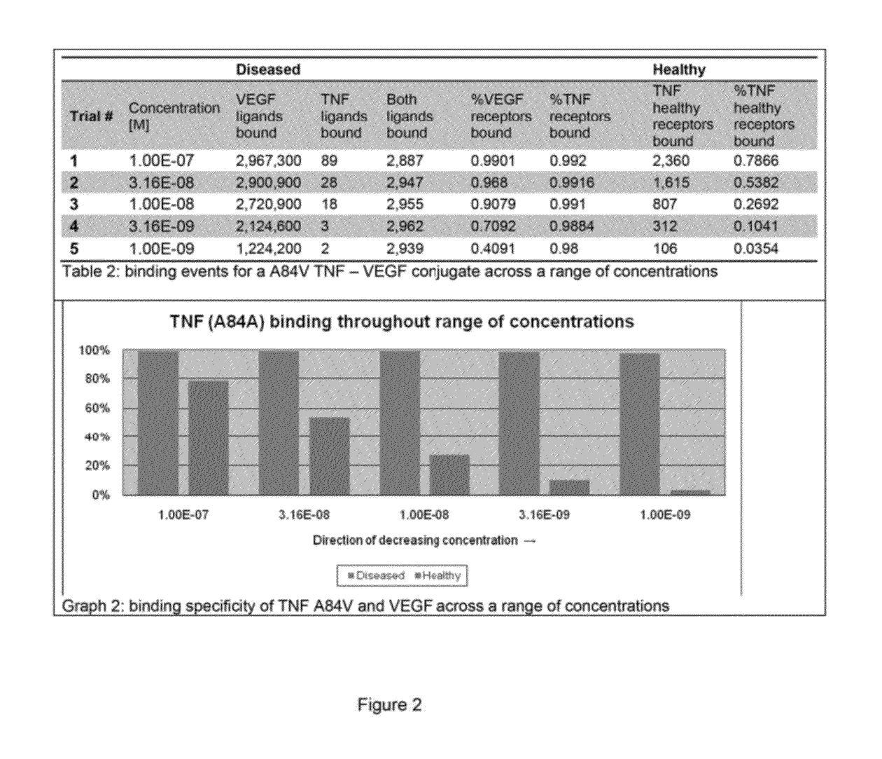

FIG. 2 is a table (Table 2) and accompanying graph (Graph 2) presenting calculated binding events for a A84V TNF-VEGF conjugate across a range of concentrations of A84V TNF-VEGF.

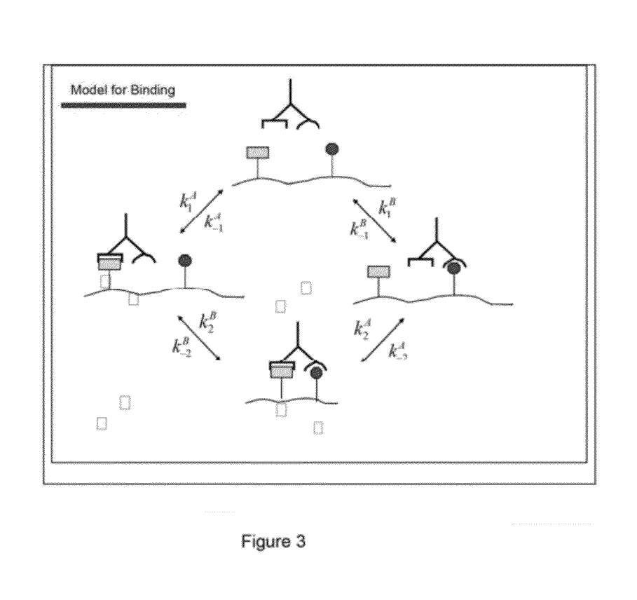

FIG. 3 is a graphic depiction of Perelson's binding model for binding of a bispecific ligand where k₁ indicates forward reaction rates, k₋₁ indicates reverse reaction rates, k₂ is the crosslinking constant and k₋₂ is the reverse reaction rate when one arm is bound

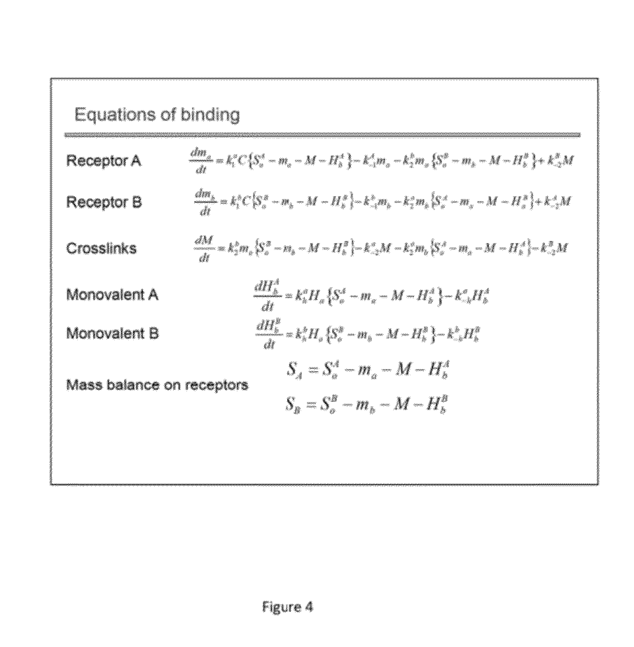

FIG. 4 shows equations based on the Perelson binding model modified to account for monovalent haptens. In these equations m is the number of single bound ligands, M is the number crosslinked ligands, k₁ is the forward binding rate, k₋₁ is the reverse binding rate, k₂ is the forward crosslink rate, k₋₂ is the reverse crosslink rate, C is the ligand concentration, S is the number of receptors, and H is the number of monovalent haptens, and a notation of a or b denotes binding of the ‘a’ arm or the ‘b’ arm.

### DETAILED DESCRIPTION

In one aspect, the present invention is adapted to planning new therapeutic radiotherapy regimens for mammals and/or particular individual mammals for particular solid tumors using rolling microspheres adapted to carry a radionuclide, that permit a more comprehensive and/or systematic coverage of radiotherapeutic treatment of vascularized tumor sites. “Microspheres” as defined above, refer to small synthetic spherical units that can have various molecules conjugated thereto, such as, but not limited to antibodies, amino acids, and therapeutic molecules. Size parameters optionally include sizes adapted for passive targeting.

### Radiotherapy—Reduced Systemic Exposure to Radionuclides

Radionuclide-bearing rolling-ligand-coated microspheres, for example paramagnetic microspheres, administered upstream from a tumor diminish the need to administer a the dose of radionuclides that is required to expose all the regions of the tumor targeted for radiotherapy. The uptake of the spheres is faster relative to antibodies used for radioimmunotherapy and each sphere by rolling through the tumor spends longer in the tumor and enhances the coverage and resulting therapeutic benefit attained on a per radionuclide basis. The spheres may also treat multiple sites of metastasis located downstream from the point of injection to which they will make their way after rolling through the first tumor site ‘visited’. For example, the ligands (PSGL-1 or sialyl-Lewis-X, optionally combined with antibody fragements or other ligands) cause the sphere to bind to the endothelium and the spheres roll slowly as the blood flow in the blood vessels push it along. Achieved rolling rates, for example, 1 to 10 microns per second, in particular 2-3 microns per second cause the sphere to reside on an endothelial cell for 1 to 5 seconds. The radionuclides emit energy that destroy the nearby cancer cells. Dosimetry can be calculated according to well know methods based on the dose distribution over the radius over which the emission damages cells having regard to the nature of the emitter. Alpha emitters are potent killers within a short range and alpha emitters can optionally (for example where damage to an intact endothelial cell wall is not required for prospective targeted deliver of microspheres progress a therapeutic regimen) be combined with beta emitters which emit energies at longer ranges suited to emssions solely from the vasculature (microspheres sized for passage into fenestrations in the tumor—passive targeting—exploit alpha emitters more than larger microspheres). For example, some alpha particles travel 50 to 80 microns. Accordingly, a microsphere adapted to roll at 1 micron per second enables each cell along the rolling path to be within range of a single radionuclide for approximately one minute. Monte carlo simulations can be employed to determine the average energy that will be emitted within the range over which the emission acts. For an alpha emitter a dose point kernel function may be used (see Zhu X, et al. Solid-tumor radionuclide therapy dosimetry: new paradigms in view of tumor microenvironment and angiogenesis. Med Phys. 2010 June; 37(6):2974-84. Janicki C. et al. Accurate determination of dose-point-kernel functions close to the origin using Monte Carlo simulations. Med Phys 2004 April; 31(4):814-8; Kassis A I. Therapeutic radionuclides: biophysical and radiobiologic principles. Semin Nucl Med. 2008 September; 38(5):358-66). According to foregoing dosing strategy, a single radionuclide can emit its energy over a substantial portion of the length of a post-capillary venule.

Rolling microspheres containing a MR contrast material (without carrying a radionuclide) analogous to nanoparticles containing a MR contrast material, and antibodies conjugated to conjugated to cross-linked iron oxide nanoparticles, enables MR imaging to be used to determine the presence of rolling ligand receptors and the average residence time of the microspheres within tumors targeted for therapy (Reynolds Radermacher K A, et al. In vivo detection of inflammation using pegylated iron oxide particles targeted at E-selectin: a multimodal approach using MR imaging and EPR spectroscopy. Invest Radiol. 2009 July; 44(7):398-404; Wyss C, et al. Molecular imaging by micro-CT: specific E-selectin imaging. Eur Radiol. 2009 October; 19(10):2487-94; Reynolds P R, et al. Detection of vascular expression of E-selectin in vivo with MR imaging. Radiology. 2006 November; 241(2):469-76. The MICAD Research Team. H18/7 F(ab′)2 E-selectin monoclonal antibody conjugated to cross-linked iron oxide nanoparticles. 2007 Mar. 26 Molecular Imaging and Contrast Agent Database [Internet]. Bethesda (MD): National Center for Biotechnology Information; Chapon C, et al. Imaging E-selectin expression following traumatic brain injury in the rat using a targeted USPIO contrast agent. MAGMA. 2009 June; 22(3):167-74)

Due to relatively (relative to monovalent or bivalent radioligands for example radioimmunoligands) efficient uptake of the microspheres by the tumor vessels attributable to multivalent binding, and the span (time and distance) over which a single radionuclide can emit damaging energy, alpha or beta emitters carried by rolling microspheres may be relatively damaging on a per radionuclide basis and attenuated side effects attributable to smaller injected doses (for example 1% to 70% of the dose) are attainable. Radial dose distributions based on residence of the emitter within the lumen of the vasculature may be calculated.

Optionally, the microspheres may be paramagnetic or superparamagnetic. Optionally, the blood carrying the rolling microspheres is passed through a magnetic sphere removal system. In one embodiment a cannula accommodating blood flow rates of the blood vessel into which it is inserted e.g the femoral artery, branches into smaller diameter conduits to which a magnetic field is applied to cause the spheres to separate from the blood based at a rate given by the formula described below according to Chen et al., Three-dimensional modeling of a portable medical device for magnetic separation of particles from biological fluids, Phys Med Biol. 2007 Sep. 7; 52(17):5205-18. Efficiencies of removal established using an apparatus based on this model enable a vast majority of the microspheres to be removed, which further reduces the toxicity of already diminished systemic exposure required to achieve standard tumoral therapeutic dosimetries.

A wide variety of microsphere core technologies and surface functionization technologies may be used to prepare rolling microspheres that are functionalized for coating rolling ligands onto a microsphere or have magnetic properties e.g. superparamagnetic and paramagnetic iron oxide spheres adapted for removal from the body based on a magnetic field—or non-magnetic rolling microspheres which may also be removed by using flow chambers adapted for such removal, for example, wherein the surface numbers of rolling ligand receptors are optionally greater relative to the neo-vascular tissue of a tumor. Notably, EP 2123269 relates to functionalized nanoparticles used to inhibit selectin mediated cell adhesion comprise a core (in the instant case chosen to be gold), a shell coating said core, formed by a monolayer of a linker molecule, and an acyclic or carbocyclic sulfated amino alcohol linked to said linker molecule via a peptide bond. This technology exemplifies a variety of non-toxic surface chemistries that may be selected especially where the microsphere is not used in an isolated limb or organ perfusion and/or not removed. Additionally, as described herein, functionalization can be specifically geared to linking more than one type of molecule to the surface of the microsphere, for example, the nanoparticle can be adapted to bear a mosaic surface pattern of multiple functional groups such as —COOH, —NH₂ and —OCH₃, for example in the manner described in WO 2010/042555 (see also WO 2008/034675) Nanoparticle technologies and functionalization well suited to MRI or other imaging technologies for determining the rolling ligand receptor (e.g. selectins) density on endothelial cells and observe residence time in a diseased tissue rolling include nanoparticles having a suitable superparamagnetic iron oxide suitable core and a coating or shell comprising a silane functionalized zwitterionic moiety (see WO 2010/076237). Magnetic nanoparticles having improved magnetic properties are well described (see for example U.S. Pat. No. 7,691,285).

Functionalization technologies include those that are specifically adapted for more precise quantification of loading (see WO 2010/040074, U.S. Pat. No. 7,507,530, US 2005/0208142, US 2007/0134679 for example for loading radionuclides) and patterned surfaces, for example where a silica surface is functionalized for controlled loading (see, Liong M, Lu J, Kovochich M, Xia T, Ruehm S G, Nel A E, Tamanoi F, Zink J I.

Multifunctional inorganic nanoparticles for imaging, targeting, and drug delivery. ACS Nano. 2008 May; 2(5):889-96. Rosenholm J M, Sahlgren C, Linden M. Towards multifunctional, targeted drug delivery systems using mesoporous silica nanoparticles—opportunities & challenges. Nanoscale. 2010 Aug. 23; those where the loading is more simply a function of rolling ligand concentrations used in the functionalization step. Functionalization for magnetic and non-magnetic nanoparticles include a variety of chemistries described in the documents referenced herein (McCarthy J R. et al. Adv Drug Deliv Rev 2008 Aug. 17 60(11) p 1241-1251; Ito A. et al. Journal of Bioscience and Bioengineering (2005) v. 100 p. 1-11; Veiseh O, et al. Design and fabrication of magnetic nanoparticles for targeted drug delivery and imaging. Adv Drug Deliv Rev. 2010 Mar. 8; 62(3):284-304. Gu F. et al., Methods Mol Biol. 2009; 544:589-98; Gu F. et al. Proc Natl Acad Sci USA. 2008 Feb. 19; 105(7):2586-91; Brault N D, et al. Biosens Bioelectron. 2010 Jun. 15; 25(10):2276-82; Bagwe RP. et al. Langmuir 2006 Apr. 25; 22(9):4357-62. Chanana M, Mao Z, Wang D. Using polymers to make up magnetic nanoparticles for biomedicine. J Biomed Nanotechnol. 2009 December; 5(6):652-68; Gupta A K, et al., Recent advances on surface engineering of magnetic iron oxide nanoparticles and their biomedical applications. Nanomedicine (Lond). 2007 February; 2(1):23-39. Babincova M, Babinec P. Magnetic drug delivery and targeting: principles and applications. Biomed Pap Med Fac Univ Palacky Olomouc Czech Repub. 2009 December; 153(4):243-50. Gazeau F, et al. Nanomedicine (Lond). 2008 December; 3(6):831-44; Frey N A, et al. Chem Soc Rev. 2009 September; 38(9):2532-42; Lin M M, et al. IEEE Trans Nanobioscience. 2008 December; 7(4):298-305; Jun Y W, et al. Acc Chem Res. 2008 February; 41(2):179-89; Liu Y, et al., Int J. Cancer. 2007 Jun. 15; 120(12):2527-37; Xu C, Sun S. Superparamagnetic nanoparticles as targeted probes for diagnostic and therapeutic applications. Dalton Trans. 2009 Aug. 7; (29):5583-91; Jia H, et al. Nanomedicine (Lond). 2009 December; 4(8):951-66; Peng X H, et al. Int J Nanomedicine. 2008; 3(3):311-21; Gupta A K et al. Synthesis and surface engineering of iron oxide nanoparticles for biomedical applications. Biomaterials. 2005 June; 26(18):3995-4021; Al-Jamal W T, et al. Liposome-nanoparticle hybrids for multimodal diagnostic and therapeutic applications. Nanomedicine (Lond). 2007 February; 2(1):85-98; McBain S C, et al. Int J Nanomedicine. 2008; 3(2):169-80. McCarthy J R, et al. Nanomedicine (Lond). 2007 April; 2(2):153-67; Latorre M, et al. Applications of magnetic nanoparticles in medicine: magnetic fluid hyperthermia. P R Health Sci J. 2009 September; 28(3):227-38. Nune S K, et al. Expert Opin Drug Deliv. 2009 November; 6(11):1175-94; Horák D, et al. Preparation and properties of magnetic nano- and microsized particles for biological and environmental separations. J Sep Sci. 2007 July; 30(11):1751-72; Liang X J, et al. Curr Drug Metab. 2008 October; 9(8):697-709. Levine D H, et al. Polymersomes: a new multi-functional tool for cancer diagnosis and therapy. Methods. 2008 September; 46(1):25-32; Shokeen M, et al. Synthesis, in vitro and in vivo evaluation of radiolabeled nanoparticles. Q J Nucl Med Mol Imaging. 2008 September; 52(3):267-77; Shubayev V I, et al. Magnetic nanoparticles for theragnostics. Adv Drug Deliv Rev. 2009 Jun. 21; 61(6):467-77; Polyak B, et al. Expert Opin Drug Deliv. 2009 January; 6(1):53-70; Huo Q, et al. J Am Chem Soc. 2006 May 17; 128(19):6447-53; Zhang R, Langmuir. 2009 Sep. 1; 25(17):10153-8; Lin M M, et al. Surface activation and targeting strategies of superparamagnetic iron oxide nanoparticles in cancer-oriented diagnosis and therapy. Nanomedicine (Lond). 2010 January; 5(1):109-33; Namdeo M, et al. J Nanosci Nanotechnol. 2008 July; 8(7):3247-71; Wang H, et al. Expert Opin Drug Deliv. 2009 July; 6(7):745-68; Wang X, et al. Exp Biol Med (Maywood). 2009 October; 234(10):1128-39.) (the contents of which are incorporated by reference) including amine functionalization (WO2003/057175), water soluble hydroxysilyl or alkoxysilyl functionalized polymers (see US 2009/0041673), cores having a silica based shell for functionalization (see for example WO 2009/029870, WO 2009/031859) functionalization based on avidin-biotin interactions (see U.S. Pat. No. 6,855,239, U.S. Pat. No. 5,252,743, U.S. Pat. No. 4,282,287) or click chemistry (see US 2009/097609) or providing a functionalized PEG polymer and coupling the targeting agent to the PEG polymer (see WO 2010/005740) (see also WO 2008/147481, WO 2008/048271, WO 20014/0067503, WO 2009/097319, U.S. Pat. No. 7,547,473m US 2010/0209352, US 2010/0092364, US 2010/0092364). The term “functionalized microsphere” as used herein is meant to refer to a nanoparticle (as explained above the prefix micro is not meant to discriminate between particles that are less than 1 micrometer in size and those that are greater in size) that is provided with (or coordinated with) functional groups or molecules that allow the binding to a specific target, such as a protein, preferably a specifically selected protein, such as a selectin, e.g. an L-, P-, or E-selectin, or a nanoparticle that is provided with functional groups or molecules that increase the stability of the nanoparticle in a given environment, e.g. by preventing aggregation.

The therapeutic coated or contained within the microspheres of the present invention is preferably a radionuclide, and the sphere may additionally carry a protein, peptide or chemotherapeutic (for example, to modulate binding to endothelial cells or to affect the surface expression of rolling ligand receptors or pericellular perfusion of adjunct therapeutics etc). The microspheres may be sized to both roll and also penetrate fenestrations in the tumor vasculature (approx. 0.1 microns) The radionuclide can be a beta-emitter (such as, but not limited to, ⁸⁹Sr, ⁹⁰Sr, ³²P) and/or an alpha-emitter (such as, but not limited to, ²⁰⁹Bi, ²¹⁰Po, ²¹⁵At, ²¹⁸Rn, ²²¹Fr, ²²³Ra, ²²⁵Ac, ²²⁷Th, ²³¹Pa, ²³³U, ²³⁷Np, ²³⁸Pu, ²⁴¹Am, ²⁴⁴Cm, ²⁴⁹Cf) with a half-life suited to the tumor residence time and recirculation frequency of the rolling microsphere and depending on whether the sphere is removed the extent of exposure.

In one embodiment, the quantity of rolling ligands is approximately matched to an expected or estimated quantity of rolling ligand receptors on the surface of endothelial cells. The method optionally comprises the step of using an experimental apparatus and/or a mathematical model and/or animal model and/or ex vivo perfusion model to predict the approximate rate of rolling of the sphere, for example a parallel plate flow chamber or other similar device and algorithms well known to those skilled in the art (see for example, Wiese G, Barthel S R, Dimitroff C J. Analysis of physiologic E-selectin-mediated leukocyte rolling on microvascular endothelium. J Vis Exp. 2009 Feb. 11; (24). pii:1009 and Ohkhouchi K et al. Cancer Research March 1990, v50 1640-1644; Siakawa A. et al. October 1996, Pharmaceutical Research v. 13(10) p 1438; Imoto H. et al. Aug. 15, 1992 Cancer Research 52, p 4396-4401; WO 2007/082742). The execution of isolated perfusions and the planning and monitoring of and individual dosimetry is well described in the art (Sreeramoju P, Libutti S K. Strategies for Targeting Tumors and Tumor Vasculature for Cancer Therapy. Adv Genet. 2010; 69C:135-152. Germain M A, Bonvalot S, Rimareix F, Missana C M. [Locally advanced soft-tissue sarcomas. An innovating triad to avoid amputation: isolated limb perfusion, TNFalpha, and free microsurgical flap]. Bull Acad Natl Med. 2010 January; 194(1):51-65; Van Schil P E, Furrer M, Friedel G. Locoregional therapy. J Thorac Oncol. 2010 June; 5(6 Suppl 2):5151-4. Rossi C R, Pasquali S, Mocellin S, Vecchiato A, Campana L G, Pilati P, Zanon A, Nitti D. Long-Term Results of Melphalan-Based Isolated Limb Perfusion With or Without Low-Dose TNF for In-Transit Melanoma Metastases. Ann Surg Oncol. 2010 Apr. 29. Alexander H R Jr, Butler C C. Development of isolated hepatic perfusion via the operative and percutaneous techniques for patients with isolated and unresectable liver metastases. Cancer J. 2010 March-April; 16(2):132-41. Divoli A, Chiavassa S, Ferrer L, Barbet J, Flux G D, Bardiés M. Effect of patient morphology on dosimetric calculations for internal irradiation as assessed by comparisons of Monte Carlo versus conventional methodologies. J Nucl Med. 2009 February; 50(2):316-23. Gabriel M, Andergassen U, Putzer D, Kroiss A, Waitz D, Von Guggenberg E, Kendler D, Virgolini I J. Individualized peptide-related-radionuclide-therapy concept using different radiolabelled somatostatin analogs in advanced cancer patients. Q J Nucl Med Mol Imaging. 2010 February; 54(1):92-9. Dewaraja Y K, Schipper M J, Roberson P L, Wilderman S J, Amro H, Regan D D, Koral K F, Kaminski M S, Avram A M. 131I-tositumomab radioimmunotherapy: initial tumor dose-response results using 3-dimensional dosimetry including radiobiologic modeling. J Nucl Med. 2010 July; 51(7):1155-62. Velasques De Oliveira S M, Julião L M, Sousa W O, Mesquita S A, Santos M S. Methodology for radionuclides quantification through “in vitro” bioassay. Cell Mol Biot (Noisy-Ie-grand). 2010 May 10; 56(2):41-3. Keenan M A, Stabin M G, Segars W P, Fernald M J. RADAR realistic animal model series for dose assessment. J Nucl Med. 2010 March; 51(3):471-6; Ho C L, Chen L C, Lee W C, Chiu S P, Hsu W C, Wu Y H, Yeh C H, Stabin M G, Jan M L, Lin W J, Lee T W, Chang C H. Receptor-binding, biodistribution, dosimetry, and micro-SPECT/CT imaging of 111In-[DTPA(1), Lys(3), Tyr(4)]-bombesin analog in human prostate tumor-bearing mice. Cancer Biother Radiopharm. 2009 August; 24(4):435-43. Chang C H, et al. Comparative dosimetric evaluation of nanotargeted (188)Re-(DXR)-liposome for internal radiotherapy. Cancer Biother Radiopharm. 2008 December; 23(6):749-58. Vicini P, Brill A B, Stabin M G, Rescigno A. Kinetic modeling in support of radionuclide dose assessment. Semin Nucl Med. 2008 September; 38(5):335-46; Stabin M G, Brill A B. State of the art in nuclear medicine dose assessment. Semin Nucl Med. 2008 September; 38(5):308-20. Cittanti C, Uccelli L, Pasquali M, Boschi A, Flammia C, Bagatin E, Casali M, Stabin M G, Feggi L, Giganti M, Duatti A. Whole-body biodistribution and radiation dosimetry of the new cardiac tracer 99 mTc-N-DBODC. J Nucl Med. 2008 August; 49(8):1299-304) and include dosimetric calculations for delivery of targeted and untargeted liposomes carrying radionuclides.

The present invention additionally provides for a method of delivering a therapeutic to a tumor, including the step of administering a targeted immune modulator in advance of and/or contemporaneously with delivery of the microspheres having a therapeutic coated thereon to a patient, rolling the microspheres in tumor vasculature, and simultaneously releasing the therapeutic dose in the tumor vasculature.

The microspheres are coated with PSGL-1 or a sialyl-Lewis-X ligand that mediates binding (CD62e) and optionally also other ligands that bind to vessel targets e.g ICAM or neo-vasculature specific ligand binding moieties (an antibody fragment that binds to a target expressed primarily on endothelial cells of tumor neo-vasculature). P-selectin glycoprotein ligand-1 (PSGL-1) recombinantly made in CHO or other cells for the proper glycosylation can be made according to techniques well know to those skilled in the art (see Glycosylation engineering in Chinese hamster ovary cells using tricistronic vectors, Journal Biotechnology Letters, Issue Volume 22, Number 1/January, 2000 Publisher Springer Netherlands, ISSN 0141-5492.) and can be loaded onto the microspheres to achieve rolling (see for example, P-selectin glycoprotein ligand-1 supports rolling on E- and P-selectin in vivo, Norman et al. Blood Journal 2000 96: 3585-3591). The effect of ligand loading and shear force has on the rate of rolling has been extensively studied by Hammer D A. et al. (see refs) and can be readily modelled empirically. Moreover, reproducible models to confirm expectations of the model. (see for example, Wiese G, Barthel S R, Dimitroff C J. Analysis of physiologic E-selectin-mediated leukocyte rolling on microvascular endothelium. J Vis Exp. 2009 Feb. 11; (24). pii:1009) have been carefully documented and methods of stimulating endothelial cell lines with inflammatory cytokines for use in the model are known to persons skilled in the art. A number of models reliably predict the effect of shear, ligand affinity, and numbers of ligands loaded on the microspheres (see Hammer et al. and Example 1; see also 1: Sperandio M, Pickard J, Unnikrishnan S, Acton S T, Ley K. Analysis of leukocyte rolling in vivo and in vitro. Methods Enzymol. 2006; 416:346-71. Pospieszalska M K, Ley K. Dynamics of Microvillus Extension and Tether Formation in Rolling Leukocytes. Cell Mol Bioeng. 2009; 2(2):207-217. Fang Y, Wu J, McEver R P, Zhu C. Bending rigidities of cell surface molecules P-selectin and PSGL-1. J Biomech. 2009 Feb. 9; 42(3):303-7. Pospieszalska M K, Zarbock A, Pickard J E, Ley K. Event-tracking model of adhesion identifies load-bearing bonds in rolling leukocytes. Microcirculation. 2009 February; 16(2):115-30. Epub 2008 Oct. 16. Erratum in: Microcirculation. 2009 November; 16(8):781. Yang J, Furie B C, Furie B. The biology of P-selectin glycoprotein ligand-1: its role as a selectin counterreceptor in leukocyte-endothelial and leukocyte-platelet interaction. Thromb Haemost. 1999 January; 81(1):1-7. Jadhav S, Eggleton C D, Konstantopoulos K. A 3-D computational model predicts that cell deformation affects selectin-mediated leukocyte rolling. Biophys J. 2005 January; 88(1):96-104. Pappu V, Doddi S K, Bagchi P. A computational study of leukocyte adhesion and its effect on flow pattern in microvessels. J Theor Biol. 2008 Sep. 21; 254(2):483-98). and the numbers of rolling ligand receptors (selectins) on endothelial cells (approximate numbers of which are well known to those skilled in the art and empirically ascertainable for a specific type of endothelial cells (by experimental immunomodulation), from banks of tumor samples or an individual's tumor by biopsy and assays well known to those skilled in the arts of immunoassays and immunohistochemistry. Endothelial cells from tumor biopsy material can also be isolated by laser capture micro-dissection and a variety of immunohistochemical or FACS analysis can be employed to count endothelial cell ligand (CAM e.g. selectin) numbers depending on the source of the material and the numbers of cells.

For example, ligand coated microspheres can be prepared, and receptor and ligand densities can be determined as outlined by Norman et al (P-selectin glycoprotein ligand-1 supports rolling on E- and P-selectin in vivo, Norman et al. Blood Journal 2000 96: 3585-3591).

The ligand coated spheres should bind diseased endothelium cells strongly enough to withstand the dislodging hydrodynamic forces of the capillary blood but not so strong that the spheres become fixed and do not roll. Key factors that determine whether a bound sphere will dislodge, roll or bind rigidly are: shear force, ligand-receptor affinity (on-rate and off-rate), the density of ligands on the surface of the spheres and the density of receptors on the surface of the epithelium (Interplay between Rolling and Firm Adhesion Elucidated with a Cell-Free System Engineered with Two Distinct Receptor-Ligand Pairs, Eniola et al, Biophysical Journal Volume 85 Oct. 2003 2720-2731). For any given target receptor the shear force, ligand-receptor affinity, and epithelium receptor density are fixed. The parameter that remains in the operators control is the ligand density on the sphere which can be tailored to yield rolling.

Approximations of the ideal sphere ligand density and the resulting sphere velocity can be found using the Krasik and Hammer model equations for Leukocyte rolling (A Semianalytic Model of Leukocyte Rolling, Ellen F. Krasik and Daniel A. Hammer, Biophysical Journal Volume 87 Nov. 2004 2919-2930). These approximations can then be verified experimentally by measuring the number of rolling spheres and their velocities across a range of sphere ligand densities and finding the density that allows for rolling as done by Eniola et al. (Interplay between Rolling and Firm Adhesion Elucidated with a Cell-Free System Engineered with Two Distinct Receptor-Ligand Pairs, Eniola et al, Biophysical Journal Volume 85 Oct. 2003 2720-2731) MRI, among other imaging studies can be used to track the course of rolling spheres in live mammalian tissues and experimental animal models, especially models in which tumors are implanted and models of isolated perfusion known to those skilled in the art (see

Additionally, more than one type of ligand can coat the sphere. For example, in the instance where one ligand may have an affinity that is too high to permit rolling it may be used to only coat a percentage of the sphere and another (weaker) ligand may coat the remainder of the sphere surface such that the resulting coated sphere will roll. The methods of preparation of such a sphere are as outlined by Eniola et al (Interplay between Rolling and Firm Adhesion Elucidated with a Cell-Free System Engineered with Two Distinct Receptor-Ligand Pairs, Eniola et al, Biophysical Journal Volume 85 Oct. 2003 2720-2731).

Radionuclide loading on the microspheres may be assessed according to well known methods and dosimetry estimated based on residence time within the radius over which the radionuclide is effective having regard to the frequency that the microspheres effectively return to the same site in the tumor (the “circulation frequency”). The circulation frequency may be empirically determined by MRI. In one embodiment the circulation frequency is controlled by injecting the rolling microsphere into an artery upstream from the tumor, capturing the rolling microsphere therapeutic downstream from the tumor and returning the rolling microsphere (or removing it and replacing it with a another microsphere) to the arterial side of the tumor. In one embodiment, this cycle is carried out at a selected circulation frequency by removing the microsphere on the venous side and returning it to the arterial side by isolated organ, limb or regional perfusion. The residence time based on the circulation frequency may selected to achieve typical dose for a solid tumor ranges of the type and stage in question, for example from 60 to 80 Gy.

To administer the treatment, the spheres are infused into the artery blood upstream from the tumor. The spheres then bind to the epithelium and slowly roll while emitting radioactive waves that directly or indirectly cause the death of the nearby cancer cells, for example by the causing DNA damage. Optionally the organ can be isolated from the body's circulatory system to prevent exposure of the therapeutic agent to other organs as outlined in U.S. Pat. No. 6,186,146 (see also WO/2007/082742). The blood removed downstream from the tumor can be passed through a magnetic particle separator device to remove the magnetic spheres from the blood. Such a device is described by Chen et al. (A comprehensive in vitro investigation of a portable magnetic separator device for human blood detoxification, Chen H et al., Phys Med Biol. 2007 Oct. 7; 52(19):6053-72. Epub 2007 Sep. 17.). Such devices have been shown in-vitro to remove up to 90% of spheres on the first pass under human physiological and clinical conditions (A comprehensive in vitro investigation of a protable magnetic separator device for human blood detoxification, Chen et al, Phys Med Biol. 2007 Oct. 7; 52(19):6053-72. Epub 2007 Sep. 17). The capture efficiency of such devices can be calculated using 3D modeling systems such as the model proposed by Chen (Three-dimensional modeling of a portable medical device for magnetic separation of particles from biological fluids, Chen et al, Phys. Med. Biol. 52 (2007) 5205-5218, doi:10.1088/0031-9155/52/17/007) and can be used to derive functional capabilities such as the maximal acceptable rate at which the blood can be cleansed. A feasible design can clear blood at rates of about 40 ml/min (A novel human detoxification system based on nanoscale bioengineering and magnetic separation techniques, Chen et al, Medical Hypotheses (January 2007), 68 (5), pg. 1071-1079).

Optionally the blood and magnetic spheres exiting the organ can re-circulate to feed again back into the blood upstream from the tumor according to well known methods of isolated limb or organ perfusion.

The equation governing removal of a paramagnetic microsphere inside a tube to which a magnetic field is applied is:

$$Sphere velocity , {u}_{p} = u + \frac{1}{9} \frac{{μ}_{0} {R}_{p}^{2} {ω}_{f m , p}}{{η}_{B}} \frac{{M}_{f m , p}}{{H}_{1}} ∇ ( {H}_{1} \cdot {H}_{1} )$$

(Three-dimensional modeling of a portable medical device for magnetic separation of particles from biological fluids, Chen et al, Phys Med Biol. 2007 Sep. 7; 52(17):5205-18. Epub 2007 Aug. 15)

The rolling ligands may be coated on the microspheres according to methods known in the art including well methods for controlling the concentration of the rolling ligand. A variety of suitable technologies for making superparamagnetic and paramagnetic microspheres are well known.

By coating rolling microspheres with rolling ligands in a manner that allows them roll preferentially on endothelial cells of a solid tumor e.g. via a selectin (e.g using PSGL-1 or sLeX and anti-ICAM 1.56 (±0.64)×10⁵ M-1s-1 and an off-rate of 1.13 (±0.11) x10⁻⁴ s[Interplay between Rolling and Firm Adhesion Elucidated with a Cell-Free System Engineered with Two Distinct Receptor-Ligand Pairs, Eniola et al, Biophysical Journal Volume 85 Oct. 2003 2720-2731]), it is possible to safely deliver a sufficient dose of radiotherapy to well vascularized tumors. Observed bone marrow toxicity may be avoided by several strategies including injecting the rolling spheres systemically or into limbs, organs and tissues having isolated circulation (e.g. isolated limb perfusion or isolated organ perfusion) and subsequently removing them in the manner herein described (with respect to perfusion systems see also WO 2000/29046, WO 2001/003755, U.S. Pat. No. 5,817,046, EP0364799B1, EP1032453, U.S. Pat. No. 6,186,146, U.S. Pat. No. 5,069,662).

### Immune Modulation

Immune modulation may be employed to enhance the uptake of chemotherapeutics, for example chemotherapeutics that result in DNA damage or defective transcription, for example topoisomerase inhibitors. The chemotherapeutics can be delivered systemically of via passively targeted nanoparticles of up to approximately 100 nm in diameter. According to one aspect of the invention, this enhanced chemotherapeutic uptake strategy is employed together with radionuclide carrying microspheres coated with ligands that mediate rolling to augment the DNA damage caused by a radionuclide payload. As briefly described above, in one aspect the invention is directed to a system of for treating a disease, for example a solid tumor, comprising a microsphere carrying a radionuclide therapeutic and ligands that mediate rolling on inflamed endothelium/neo-vasculature and an immune modulator for increasing the endothelial surface expression of complementary ligands, to which the ligands that mediate rolling bind. Optionally, the For convenience, the ligands that mediate rolling may be referred to as “rolling ligands” and their corresponding ligands on inflamed endothelium/neo-vasculature as receptors or “rolling ligand receptors”. In one embodiment the immune modulator is targeted to the inflamed endothelium via a peptide, antibody or protein that preferentially binds to inflamed endothelium/neo-vasculature, for increasing the concentration of the immune modulator in the inflamed endothelium/neo-vasculature. A variety of targets and targeting ligands are well known to be adaptable for this purpose including receptors for VEGF (VEGF isoforms 121, 165 and 189 and constructs suitable for their expression are well known. Celec P, et al. The use of transformed Escherichia coli for experimental angiogenesis induced by regulated in situ production of vascular endothelial growth factor—an alternative gene therapy. Med Hypotheses. 2005; 64(3):505-11. Deodato B, et al. Recombinant AAV vector encoding human VEGF165 enhances wound healing. Gene Ther. 2002 June; 9(12):777-85; Cohen T, Gitay-Goren H, Neufeld G, Levi B Z. High levels of biologically active vascular endothelial growth factor (VEGF) are produced by the baculovirus expression system. Growth Factors. 1992; 7(2):131-8. Zhou Z J, et al. Cloning of expression vector for VEGF121 and VEGF165 genes encoding human vascular endothelial growth factor. Di Yi Jun Yi Da Xue Xue Bao. 2002 February; 22(2):111-3; Ferrara N, Gerber H P, LeCouter J. The biology of VEGF and its receptors. Nat Med. 2003 June; 9(6):669-76). Their coding sequences may be used for as fusion proteins with TNF or mutants thereof see particularly Yuan X, et al. Recombinant CPE fused to tumor necrosis factor targets human ovarian cancer cells expressing the claudin-3 and claudin-4 receptors. Mol Cancer Ther. 2009 July; 8(7):1906-15; see also Lyu M A, et al. The immunocytokine scFv23/TNF targeting HER-2/neu induces synergistic cytotoxic effects with 5-fluorouracil in TNF-resistant pancreatic cancer cell lines. Biochem Pharmacol. 2008 Feb. 15; 75(4):836-46; Kim S, et al. Improved expression of a soluble single chain antibody fusion protein containing tumor necrosis factor in Escherichia coli. Appl Microbiol Biotechnol. 2007 November; 77(1):99-106; Liu Y, et al. The antimelanoma immunocytokine scFvMEL/TNF shows reduced toxicity and potent antitumor activity against human tumor xenografts. Neoplasia. 2006 May; 8(5):384-93. Liu Y, et al. Recombinant single-chain antibody fusion construct targeting human melanoma cells and containing tumor necrosis factor. Int J Cancer. 2004 Feb. 10; 108(4):549-57), anti-CD105 and ant-alpaVbeta3 ligands such as antibodies, peptides and their analogues and NGR peptides that target the angiogenic endothelial cell marker aminopeptidase (see Loi M, et al. Combined targeting of perivascular and endothelial tumor cells enhances anti-tumor efficacy of liposomal chemotherapy in neuroblastoma. J Control Release. 2010 Jul. 1; 145(1):66-73. Pastorino F, et al. Targeting liposomal chemotherapy via both tumor cell-specific and tumor vasculature-specific ligands potentiates therapeutic efficacy. Cancer Res. 2006 Oct. 15; 66(20):10073-82. Corti A, et al. Immunomodulatory Agents with Antivascular Activity in the Treatment of Non-Small Cell Lung Cancer: Focus on TLR9 Agonists, IMiDs and NGR-TNF. J Oncol. 2010; 2010:732680. Curnis F, et al. Critical role of flanking residues in NGR-to-isoDGR transition and CD13/integrin receptor switching. J Biol Chem. 2010 Mar. 19; 285(12):9114-23. Di Paolo D, et al. Liposome-mediated therapy of neuroblastoma. Methods Enzymol. 2009; 465:225-49; Gregorc V, et al. Defining the optimal biological dose of NGR-hTNF, a selective vascular targeting agent, in advanced solid tumours. Eur J Cancer. 2010 January; 46(1):198-206; Pastorino F, et al. Enhanced antitumor efficacy of clinical-grade vasculature-targeted liposomal doxorubicin. Clin Cancer Res. 2008 Nov. 15; 14(22):7320-9; Curnis F, et al. Isoaspartate-glycine-arginine: a new tumor vasculature-targeting motif. Cancer Res. 2008 Sep. 1; 68(17):7073-82. Corti A, et al. The neovasculature homing motif NGR: more than meets the eye. Blood. 2008 Oct. 1; 112(7):2628-35; Spitaleri A, Mari S, Curnis F, Traversari C, Longhi R, Bordignon C, Corti A, Rizzardi G P, Musco G. Structural basis for the interaction of isoDGR with the RGD-binding site of alphavbeta3 integrin. J Biol Chem. 2008 Jul. 11; 283(28):19757-68. Bertilaccio M T, et al. Vasculature-targeted tumor necrosis factor-alpha increases the therapeutic index of doxorubicin against prostate cancer. Prostate. 2008 Jul. 1; 68(10):1105-15. Bellone M, Mondino A, Corti A. Vascular targeting, chemotherapy and active immunotherapy: teaming up to attack cancer. Trends Immunol. 2008 May; 29(5):235-41; Crippa L, et al. Synergistic damage of tumor vessels with ultra low-dose endothelial-monocyte activating polypeptide-II and neovasculature-targeted tumor necrosis factor-alpha. Cancer Res. 2008 Feb. 15; 68(4):1154-61; Marchió S, et al. Aminopeptidase A is a functional target in angiogenic blood vessels. Cancer Cell. 2004 February; 5(2):151-62).

The immune modulator may be a cytokine, for example a chemokine or a member of the interleukin family such as IL-1β and may be coated on a nanopaticle bearing a peptide, antibody or protein that preferentially binds to inflamed endothelium/neo-vasculature, for this purpose. In one embodiment the immune modulator is TNF-α. In one embodiment, the TNF-α is targeted to the tumor vasculature via a peptide, antibody or protein that preferentially binds to inflamed endothelium/neo-vasculature, for increasing the concentration of the ligand (and optionally its avidity) in the vicinity of tumor vasculature such as being linked to VEGF via a linker, optionally a flexible linker of 10 to 35 amino acids.

The binding of dual affinity ligands to a cell surface is strongly a function of the individual ligand on-rates and off-rates, the number of ligands, and the number of cell surface receptors. The design of dual affinity ligands is enhanced by computer and mathematical modeling.

A model for receptor cross-linking by ligands bearing two chemically distinct functional groups (Perelson, Alan¹ Receptor Clustering on a Cell Surface II. Theory of Receptor Cross-linking by Ligands Bearing Two Chemically Distinct Functional Groups. Mathematical Biosciences. May 1980, Vol. 49, 1-2, pp. 87-110 may be employed as shown herein.

The Perelson model assumes that the intrinsic rate of crosslinking between two membrane associated species is time-invariant, i.e. k₂^{A}=k₂^{B}=k_{c}=constant. Other authors derive a functional form for k_{c} related to the molecular diffusivities, Dᵢ, collision radius, a, and half the mean distance between the surface species in excess, b:

$${k}_{c} = \frac{2 π ( {D}_{RA} + {D}_{RB} )}{ln ( \frac{b}{a} ) - C} \qquad Eq . 1$$

The value of b is estimated from the surface density of free receptors, ρ_{R}:

$$π {b}^{2} = \frac{1}{{ρ}_{R}} = \frac{{A}_{cell}}{{n}_{R}} \qquad Eq . 2$$

or equivalently

$$b = \sqrt{\frac{1}{{πρ}_{R}}} \qquad Eq . 3$$

Importantly, when the surface density of free receptors is significantly depleted by the addition of crosslinkers, the value of b will be transient and increase with time. Hence, k_{c}, is not truly constant, but a transcendental function of receptor density that decreases as the system equilibrates. The value of the constant C that appears in the denominator of Eq. 1 is determined by the nature of an arbitrary boundary condition introduced in the derivation flux towards a sink. Published values include C=0, 0.231, 0.5, and 0.75 (Lauffenburger, Douglas. Receptors: models for binding, trafficking, and signaling. New York: Oxford University Press, 1993. pp. 151-157)

A modified version of this model that accounts for the depletion of receptors over time has been compared to Monte Carlo random walk simulations with close agreement.

TNF-α mutants suitable for use include those well known to person skilled art.

| | Fold reduction | |
|---|---|---|
| | in affinity | |
| Mutation | for TNFR-1 | Reference |
| A84V | 1428 | Van Ostade, X., et al. Eur J Biochem. |
| | | 1994; 220(3): 771-9. |
| D143Y | >50 | Van Ostade, X., et al. Eur J Biochem. |
| | | 1994; 220(3): 771-9., Loetscher, H., et |
| | | al. J Biol Chem. 1993; 268(35): 26350-7. |
| D143F | >5000 | Van Ostade, X., et al. Eur J Biochem. |
| | | 1994; 220(3): 771-9. |
| D143E | 333 | Van Ostade, X., et al. Eur J Biochem. |
| | | 1994; 220(3): 771-9. |
| D143N | >2500 | Van Ostade, X., et al. Eur J Biochem. |
| | | 1994; 220(3): 771-9. Loetscher, H., et |
| | | al. J Biol Chem. 1993; 268(35): 26350-7. |
| R32W, | 17 | Van Ostade, X., et al. Eur J Biochem. |
| E146H | | 1994; 220(3): 771-9. |
| R32W, | 17 | Van Ostade, X., et al. Eur J Biochem. |
| E146K | | 1994; 220(3): 771-9. |
| L29S | 8.5 | Van Ostade, X., et al. Eur J Biochem. |
| | | 1994; 220(3): 771-9. |

The different TNF-α mutants can be tested in combination with the targeting arm for optimal specific binding to target cells.

It is known that bone marrow endothelium expresses a higher constitutive amount of E-selectin than non-inflamed endothelium to enable CD34+ cells to interact with the bone marrow (Hidalgo, et al.). Therefore, in order to ensure selective targeting of tumor vasculature and not other sites of inflammation and bone marrow, the tumor vasculature can be pre-treated with a tumor vasculature penetration enhancer. Preferably, the tumor vasculature penetration enhancer is a dual affinity ligand that has a high affinity vascular endothelial growth factor receptor (VEGR)-1/2 targeting arm linked by a flexible linker to a lower affinity tumor necrosis factor (TNF) receptor agonist arm. This fusion protein can be most efficiently generated by linking vascular endothelial growth factor A (VEGF A) and a TNF-α mutant with a lower affinity for tumor necrosis factor receptor 1 (TNFR-1) and TNFR-2 (Cha S, et al.). Also, a mutant form of VEGF A can be used that has a significantly lower affinity for vascular endothelial growth factor receptor 2 (VEGFR-2) than for VEGFR-1, e.g. 100 fold lower—this reduces tissue factor up-regulation and reduce E-selectin shedding (Shen B., et al.; Cha S., et al.). Optionally, shedding of selectins and/or TNF receptors be attenuated by administering antagonists of enzymes responsible for shedding e.g. ADAM17

The effect of the pre-treatment is two-fold. First, it causes the microspheres to bind upstream from the capillaries, thereby increasing tumor residence time and potentiating the efficient contemporaneous delivery of therapeutics. Second, it increases vascular permeability for enhanced delivery of therapeutics.

It has been very recently demonstrated that VEGF synergistically enhances the induction of E-selectin by TNF-α (Stannard A K, et al.).

Pre-treatment of a tumor with a VEGF A—TNF mutant fusion protein enables binding to be achieved with a lower amount of sialyl-Lewis-X or PSGL-1 (loaded onto the microspheres)—an amount that would be insufficient to mediate binding on bone marrow endothelium or endothelium of other inflamed tissues (e.g. sites of arthritis); sites where the level of E-selectin or P-Selectin expression is anticipated to remain much lower due to the targeting properties of the VEGF A—TNF mutant fusion protein. The dual affinity targeting principle is that colocalization of the two targets and their contemporaneous ligation enables the affinity of the effector arm to be made lower than would otherwise be suitable for a ligand that is both a targeting moiety and an effector moiety. The result is that the functional affinity of the bispecific ligand is much higher on target cells than on non-target cells whereupon a dose suitable for the higher affinity binding is insufficient to cause an effective concentration of the bispecific ligand to accumulate in the microenvironment of non-target cells to produce deleterious side effects.

One requisite approach to loading the ligand, the formula for predicting the correct amounts of ligand, and the impact on binding is disclosed by Hammer D A, et al. Endothelia from various tumor samples can be tested to demonstrate co-localization of VEGF and TNF receptors and known mathematical formulas can be used to ensure that the optimal biasing of the biodistribution of the VEGF-TNF fusion protein relative to non-inflamed endothelium, bone marrow and putative non-tumor sites of inflammation.

According to one embodiment the microspheres as described above can be used to deliver chemotherapeutics to the tumor vasculature. A strategy for using targeted poly(lactic-co-glycolic acid) (PLGA) microspheres to tumor vasculature to deliver weak acid chemotherapeutics that have enhanced uptake in the higher pH tumor microenvironment relative to physiological ph of 7.4 has been developed with camptothecins (Gerweck L E, et al.).

The VEGF A-mutant TNF-α fusion protein can be used to increase drug penetration. Endothelia from various tumor samples can be tested to demonstrate co-localization of VEGF and TNF receptors and known mathematical formulas can be used to ensure that the optimal biasing of the biodistribution of the VEGF-TNF fusion protein relative to non-inflamed endothelium, bone marrow and putative non-tumor sites of inflammation. Receptor numbers for VEGFR1 and VEGFR2, for example, on colonic endothelium have been determined to be approximately 80,000 and 230,000 respectively (Wang D, et al.).

TNF receptor 1 is known to play an important role in vascular permeability (90) Agonistic TNFR antibodies that bind to TNFR-1 include htr-9 and htr-1 Agonistic antibodies recognizing TNFR-1 and TNFR-2 include FT2. Ferrero E, et al (supra) and Espevik T, et al. Characterization of binding and biological effects of monoclonal antibodies against a human tumor necrosis factor receptor. J Exp Med. 1990 Feb. 1; 171(2):415-26). Galloway C J, et al. Anti-tumor necrosis factor receptor and tumor necrosis factor agonist activity by an anti-idiotypic antibody. Eur J Immunol. 1992 November; 22(11):3045-8). See also Corti A., Strategies for improving the anti-neoplastic activity of TNF by tumor targeting. Methods Mol Med. 2004; 98:247-64; see also Sacchi et al. Cancer Res. 2004 Oct. 1; 64(19):7150-5).

According to one embodiment of the invention TNF or TNF mutein (see for example WO/2004/082595) is targeted to the tumor vasculature, for example via a ligand that binds to a target that is primarily expressed or over-expressed on tumor endothelium, for example VEGFR, endoglin or NGF Corti A., Strategies for improving the anti-neoplastic activity of TNF by tumor targeting. Methods Mol Med. 2004; 98:247-64; see also Sacchi et al. Cancer Res. 2004 Oct. 1; 64(19):7150-5).

The composition of the present invention can be a pharmaceutical composition and thus include a pharmaceutically acceptable carrier as described below.

The composition of the present invention is administered and dosed in accordance with good medical practice, taking into account the clinical condition of the individual patient, the site and method of administration, scheduling of administration, patient age, sex, body weight and other factors known to medical practitioners. The present invention is also directed to administering a pharmaceutically “effective amount” of a heterofunctional ligand as described herein. A pharmaceutically “effective amount” for purposes of administration of according herein is primarily determined by a concentration of the ligand geared to a achieving nanomolar concentrations in the endothelial microenvironment of the tumor vasculature and avoiding higher concentrations that would enable the relatively reduced affinity of the TNFalpha mutant to bind in effective amounts to non-targeted endothelial cells as determined by modelling and such considerations described above and as are known in the art. The amount must be effective to achieve improvement in tumor penetration and/or expression of selectins in tumor vasculature and other indicators as measures known by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants and vehicles. The heterofunctional ligands and microspheres can be administered parenterally including intravenous, intraarterially as well as by infusion techniques related to isolated tissue perfusion. The subject being diagnosed, experimented on and/or treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the maintenance of the required particle size in the case of dispersion and by the use of surfactants. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form suitable targeted nanoparticle therapeutic, diagnostic or theragnostic delivery systems aided by other adjunct molecules. Other examples of nanoparticles systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other therapeutic delivery systems, and modules are well known to those skilled in the art.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

All of the documents referred to in this specification are hereby incorporated by reference herein in their entirety.

### Example 1

Ligand coated 1 μm microspheres are prepared, and receptor and ligand densities determined, as outlined by Norman et al (P-selectin glycoprotein ligand-1 supports rolling on E- and P-selectin in vivo, Norman et al. Blood Journal 2000 96: 3585-3591). These are assessed to determine the predicted rolling velocity with reference to actual data obtained in the lab. The ligand coated spheres are prepared in order to bind diseased endothelium cells strongly enough to withstand the dislodging hydrodynamic forces of the blood in post-capillary venules but not so strong that the spheres become fixed and do not roll. Key factors that determine whether a bound sphere will dislodge, roll or bind rigidly are: shear force, antibody-receptor affinity (on-rate and off-rate), the density of ligands on the surface of the spheres and the density of receptors on the surface of the epithelium (Interplay between Rolling and Firm Adhesion Elucidated with a Cell-Free System Engineered with Two Distinct Receptor-Ligand Pairs, Eniola et al, Biophysical Journal Volume 85 Oct. 2003 2720-2731). For any given target receptor the sheer force, ligand-receptor affinity, and epithelium receptor density are fixed. The parameter that remains in the operators control is the ligand density on the sphere which can be tailored to yield rolling.

Approximations of the ideal sphere ligand density and the resulting sphere velocity are determined using the Krasik and Hammer model equations for Leukocyte rolling (A Semianalytic Model of Leukocyte Rolling, Ellen F. Krasik and Daniel A. Hammer, Biophysical Journal Volume 87 Nov. 2004 2919-2930). These approximations are then be verified experimentally by measuring the number of rolling spheres and their velocities across a range of sphere ligand densities and finding the density that allows for rolling as done by Eniola et al. (Interplay between Rolling and Firm Adhesion Elucidated with a Cell-Free System Engineered with Two Distinct Receptor-Ligand Pairs, Eniola et al, Biophysical Journal Volume 85 Oct. 2003 2720-2731).

A PSGL-1 ligand that binds to P-selectin with a forward reaction rate of 0.02 μm²/s and a reverse rate of 5 s⁻¹ (A Semianalytic Model of Leukocyte Rolling, Ellen F. Krasik and Daniel A. Hammer, Biophysical Journal Volume 87 Nov. 2004 2919-2930) can be attached to a 5 μm-radius sphere at a surface density of 48/μm². In post capillary vessels with diameters and centerline velocities of approximately 40 μm and 2.8 mm/second respectively (P-selectin glycoprotein ligand-1 supports rolling on E- and P-selectin in vivo, Norman et al. Blood Journal 2000 96: 3585-3591), anticipated shear rates range from 130-400 s⁻¹ (In a pipe, shear rate=8*fluid velocity/diameter; physiologically relevant shear rates are reported by Hammer to be 130 s⁻¹). Assuming P-selectin density of 200/μm², the Krasik model predicts velocities of 1.75-5 μm/s which closely approximates the 4-7 μm/s found experimentally (A Semianalytic Model of Leukocyte Rolling, Ellen F. Krasik and Daniel A. Hammer, Biophysical Journal Volume 87 Nov. 2004 2919-2930 see FIG. 2). Referring to FIG. 2 in Krasik et al. (supra), shown shear rates span the physiological range. Robbins et al. (Tunable leuko-polymersomes that adhere specifically to inflammatory markers, Robbins G P et al, Langmuir, Article ASAP DOI: 10.1021/Ia1017032) reports the physiologically relevant shear rate to be 130/s. According to this graph this would result in a rolling velocity of 2-5 μm/second.

Parameters used in the Krasik et al. (supra) model and an explanation of the top left legend follows:

Chosen Cell radius=5 μm

Explanation of top left legend:

λ: Equilibrium bond length

rc: Reactive compliance

σ: Bond spring constant

kr0: Intrinsic reverse reaction rate

kf0: Intrinsic forward reaction rate

nLT: ligand density (on wall surface)

### REFERENCES

1. Reilly R M. Radioimmunotherapy of solid tumors: the promise of pretargeting strategies using bispecific antibodies and radiolabeled haptens. J Nucl Med. 2006 February 47(2):196-9.
2. Goldenberg D M, et al. Advances in cancer therapy with radiolabeled monoclonal antibodies. Q J Nucl Med Mol Imaging. 2006 December 50(4):248-64.
3. Escorcia F E, McDevitt M R, VIIIa C H, Scheinberg D A. Targeted nanomaterials for radiotherapy. Nanomed. 2007 Dec. 2(6):805-15.
4. Sofou S, Kappel B J, Jaggi J S, McDevitt M R, Scheinberg D A, Sgouros G. Enhanced retention of the alpha-particle-emitting daughters of Actinium-225 by liposome carriers. Bioconjug Chem. 2007 Nov.-Dec. 18(6):2061-7.
5. Eniola A O, Rodgers S D, Hammer D A. Characterization of biodegradable drug delivery vehicles with the adhesive properties of leukocytes. Biomaterials. 2002 May 23(10):2167-77.
6. Eniola A O, Hammer D A. Artificial polymeric cells for targeted drug delivery. J Control Release. 2003 Feb. 21 87(1-3):15-22.
7. Eniola A O, Willcox P J, Hammer D A. Interplay between rolling and firm adhesion elucidated with a cell-free system engineered with two distinct receptor-ligand pairs. Biophys J. 2003 (85):2720-31.
8. Omolola Eniola A, Hammer D A. In vitro characterization if leukocyte mimetic for targeting therapeutics to the endothelium using two receptors. Biomaterials. 2005 Dec. 26(34):7136-44.
9. Andres Hidalgo, et al. Functional selectin ligand mediating human CD34+ cell interactions with bone marrow endothelium are enhanced postnatally. J Clin Invest. 110:559.
10. Shen B, et al. JBC Vol. 276, p. 5281, Feb. 16, 2001.
11. Cha S, et al. High resolution crystal structure of a human tumor necrosis factor-α mutant with low systemic toxicity. JBC Vol. 273, No. 4, pp. 2153-2160, Jan. 23, 1998.
12. Stannard A K, et al. Vascular endothelial growth factor synergistically enhances induction of E-selectin by TNF-α. Arterioscler Thromb Vasc Biol. p. 494, March 2007.
13. Wang D, et al. Am J Physiol Gastrointes Liver Physiol. 282 G1088-G1096 2002.
14. Gerwick L E, et al. Tumor pH controls the in vivo efficacy of weak acid and base chemotherapeutics. Mol Cancer Ther. 2006 May 5(5):1275-9.
15. Tong W, et al. Evaluation of PLGA microspheres as delivery system for antitumor agent-camptothecin. Drug Dev Ind Pharm. 2003 Aug. 29(7):745-56.
16. Dora C L, et al. Evaluation of antimetastatic activity and systemic toxicity of camptothecin-loaded microspheres in mice injected with B16-F10 melanoma cells. J Pharm Pharm Sci. 2006 9(1):22-31.
17. Loch-Neckel G, et al. Stealth and non-stealth nanocapsules containing camptothecin: in-vitro and in-vivo activity on B16-F10 melanoma. J Pharm Pharmacol. 2007 October 59(10):1359-64.
18. Jackson, J K, et al. The characterization of paclitaxel-loaded microspheres manufactured from blends of poly(lactic-co-glycolic acid) (PLGA) and low molecular weight diblock copolymers. Int J Pharm. 2007 Sep. 5 342(1-2):6-7.
19. Winter M E. Basic clinical pharmacokinetics. 4th ed. Philadelphia: Lippincott Williams & Wilkins; 2004.

### Example 2

One experiment was performed for a VEGF antibody conjugated with the D143 TNF mutant which has a 333-fold reduction in affinity relative to the wild type 1.9e-11 M affinity of TNF. The numbers of VEGF, TNF and crosslinked bindings were measured for a range of concentrations for 3 million VEGF receptors with 1.0 nanomolar (high) affinity and 3 thousand D143 TNF receptors with 6.33 nanomolar (low) affinity. The number of bound TNF receptors on healthy cells was also computed for comparison. For the healthy cells, the number of VEGF receptors was assumed to be zero.

### Example 3

Another experiment was performed for a VEGF antibody conjugated with the A84V mutant of TNF which has a 1428-fold reduction in affinity relative to the wild type 1.9e-11 M affinity of TNF. The numbers of VEGF, TNF and crosslinked bindings were measured for a range of concentrations for 3 million VEGF receptors with 1.0 nanomolar (high) affinity and 3 thousand A84V TNF receptors with 27.13 nanomolar (low) affinity. The number of bound TNF receptors on healthy cells was also computed for comparison. For the healthy cells, the number of VEGF receptors was assumed to be zero.

FIGS. 1 and 2 illustrate the results of two simulation experiments as described above. Analysis of the results from both simulations show that as the concentration drops, the positive effect of dual affinity ligands becomes more pronounced because the percentage of bound B receptors on diseased cells stays high whereas the percentage of bound B receptors on healthy cells drops.

What is claimed is:

1. A composition comprising microspheres including rolling ligands that enhance binding in tumor vasculature and a therapeutic coated thereon.

2. The composition of claim 1, wherein said microspheres are chosen from the group consisting of superparamagnetic and paramagnetic microspheres.

3. The composition of claim 1, wherein said rolling ligands are further defined as ligands for mediating rolling on inflamed endothelium and optionally include neo-vasculature specific ligand binding moieties.

4. The composition of claim 3, wherein said rolling ligands are further defined as ligands for interacting with E-selectin, L-Selectin or P-Selectin.

5. The composition of claim 4, wherein said ligand means are further defined as sialyl-Lewis-X or PSGL-1.

6. The composition of claim 1, wherein said therapeutic is a radionuclide.

7. A composition of matter comprising a heterofunctional ligand including a neo-vascular targeting ligand and an TNFR-1 or TNFR-2 agonist.

8. A composition of matter according to claim 7, wherein the heterofunctional ligand is a bispecific antibody.

9. A composition of matter according to claim 7, wherein the agonist is a TNFR-1 agonist having an affinity for TNFR-1 that is 10 to 5000 fold lower than wild-type TNFα.

10. A composition of matter according to claim 7, wherein the neo-vascular targeting ligand colocalizes with the TNFR agonist, optionally as demonstrated by FRET, co-immunoprecipitation or a competition binding experiment.
